(19) United States
(12) Patent Application Publication
(10) Pub. No.: US 2008/0261326 A1
(43) Pub. Date: Oct. 23, 2008

(54) Drop-on-demand manufacturing of diagnostic test strips

(76) Inventors: Christie Dudenhoefer, Corvallis, OR (US); Kenneth Ward, Corvallis, OR (US); Joseph W. Dody, Corvallis, OR (US); Isaac Farr, Corvallis, OR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/738,923

(22) Filed: Apr. 23, 2007

(51) Int. Cl. G01N 33/00; G01N 1/00

(52) U.S. Cl. 436/174; 422/73

(57) ABSTRACT

A drop-on-demand system for manufacturing diagnostic test strips includes a drop-on-demand mechanism, and a drop volume feedback subsystem and/or a vision subsystem. The drop-on-demand mechanism dispenses one or more reagents on the diagnostic test strips in one or more desired volumes and at one or more desired locations. The drop volume feedback subsystem control volumes of the reagents dispensed by the drop-on-demand mechanism so that the drop-on-demand mechanism dispenses the reagents on the diagnostic test strips in the desired volumes. The vision subsystem aligns the drop-on-demand mechanism in relation to the diagnostic test strips so that the drop-on-demand mechanism dispenses the reagents on the diagnostic test strips at the desired locations.

### RELATED APPLICATIONS

The present patent application is related to the following cofiled patent applications: “Sensing of fluid ejected by drop-on-demand nozzles,” having inventors Isaac V. Farr et al., filed on ______, and assigned Ser. No. ______; and, “Printing control,” having the inventor David R. Otis, Jr., filed on ______, and assigned Ser. No. ______.

### BACKGROUND

Diagnostic test strips are used in a variety of medical and other applications. For example, a glucose monitoring diagnostic test strip enables a person inflicted with diabetes to easily test his or her current glucose level. The person places a drop of blood on the reagent on the diagnostic test strip. Based on how the blood reacts with the reagents, the resulting mixture may change in color or generate an electrical current. Based on this response, the person can determine his or her current blood glucose level.

Current manufacturing of diagnostic test strips, however, can be imprecise. For example, micropipettes can be used to dispense the needed reagents on the diagnostic test strips. However, micropipettes can only accurately dispense reagents on diagnostic test strips down to microliter levels of volumetric precision, and with limited control over the shape of the dispensed reagents. Some types of diagnostic test strips may require more precise dispensing of reagents, or may benefit from reagents patterned in complex shapes. Micropipetting and other current processes for making diagnostic test strips can also suffer from reagent wastage problems, as well as other problems that can increase manufacturing cost and reduce manufacturing flexibility.

### BRIEF DESCRIPTION OF THE DRAWINGS

FIG. 1 is a diagram of a representative diagnostic test strip 100, according to an embodiment of the invention.

FIGS. 2A and 2B are diagrams depicting a number of diagnostic test strips, according to different embodiments of the invention.

FIG. 3 is a block diagram of a drop-on-demand system for manufacturing diagnostic test strips, according to an embodiment of the invention.

FIGS. 4A and 4B are diagrams showing how a drop-on-demand mechanism can dispense reagents onto a diagnostic test strip, according to an embodiment of the invention.

FIG. 5 is a diagram showing how a drop volume feedback subsystem can verify and control volumetric output of reagents, according to an embodiment of the invention.

FIGS. 6A, 6B, and 6C are diagrams showing how a vision subsystem can compensate for variations between and within a drop-on-demand mechanism and a diagnostic test strip, according to an embodiment of the invention.

FIG. 7 is a flowchart of a rudimentary method, according to an embodiment of the invention.

### DETAILED DESCRIPTION OF THE DRAWINGS

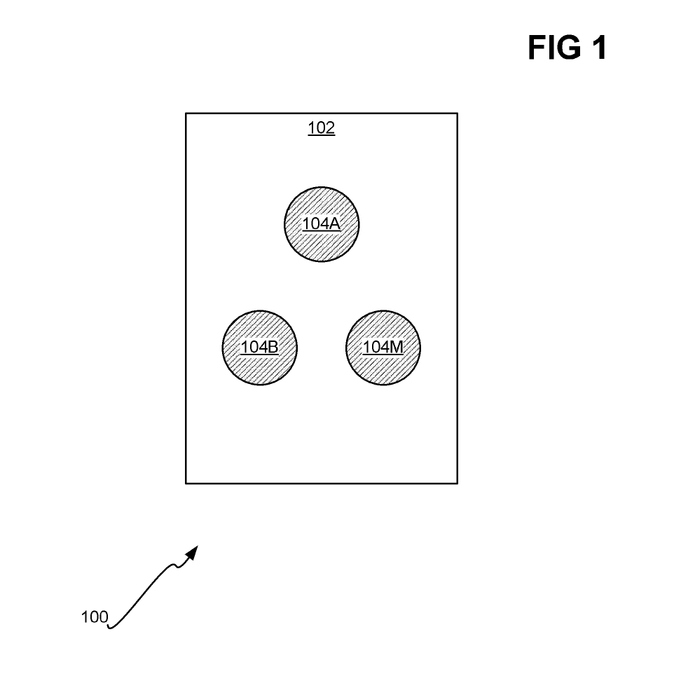

FIG. 1 shows a representative diagnostic test strip 100, according to an embodiment of the invention. The diagnostic test strip 100 includes a substrate 102 on which a number of reagents 104A, 104B, . . . , 104M, collectively referred to as the reagents 104, have been placed in a drop-on-demand manner, as is described in more detail later in the detailed description. The diagnostic test strip 100 may include other components, in addition to those depicted in FIG. 1, such as electrodes, as can be appreciated by those of ordinary skill within the art. The substrate 102 may be an absorptive or a non-absorptive substrate, and may be a polymeric substrate, or another type of substrate, such as paper, metal, or another type of substrate. The substrate may also have three-dimensional features in one embodiment of the invention.

The reagents 104 may be the same or different reagents. The locations at which the reagents 104 are placed on the substrate 102 can be precisely controlled. Additionally, the amount of each reagent 104 that is placed on the substrate 102 can be precisely controlled. Furthermore, the morphology, including the thickness, shape and/or texture, of each of the reagents 104 can be precisely controlled. While three locations at which the reagents 104 have been placed on the substrate 102 are depicted in FIG. 1, there may be more or less of such locations in other embodiments of the invention. In one embodiment, the reagents may be adjacent to or overlap one another.

Examples of types of reagents include proteins, enzymes, such as glucose oxidase and glucose dehydrogenase, anti-bodies, DNA, RNA, oligomers, small molecules, as well as active pharmaceutical ingredients, and thromboplastin reagent, among other types of diagnostic reagents and supporting agents, such as buffers, surfactants, and polymers. As such, the purpose of the diagnostic test strip 100 may be for glucose monitoring, in which a person places a drop of blood on the test strip 100, which reacts with the reagents 104 in such a way so that the glucose level of the person can be determined. As another example, the purpose of the diagnostic test strip 100 may be for fertility diagnostic purposes, to determine, for instance, whether a woman is pregnant or not. Other applications for the diagnostic test strip 100 include determining blood clotting rate (i.e., INR), testing for the presence of commonly abused drugs, determining whether ovulation is occurring, determining whether heart disease is present, as well as testing for HIV or other infectious diseases, among other types of applications.

The manner by which the diagnostic test strip 100 is used for its intended purpose is not limited by embodiments of the invention. In one embodiment, for instance, a sample containing the substance of interest is placed on the diagnostic test strip 100 and reacts with the reagents 104 in such a way as to change color, to provide visual indication as to the amount, or presence, of this substance, potentially leading to diagnosis of a specific condition. In another embodiment, the resulting mixture of the substance-containing sample placed on the test strip 100 and the reagents 104 is tested in a different way, such as by using a meter that measures an electrical property, to provide for quantification of the substance. In general, therefore, the diagnostic test strip 100 is used in such a way that a sample containing a substance is placed on the test strip 100, where the substance reacts with the reagents 104 so that proper quantification of this substance or diagnosis of a condition can be achieved.

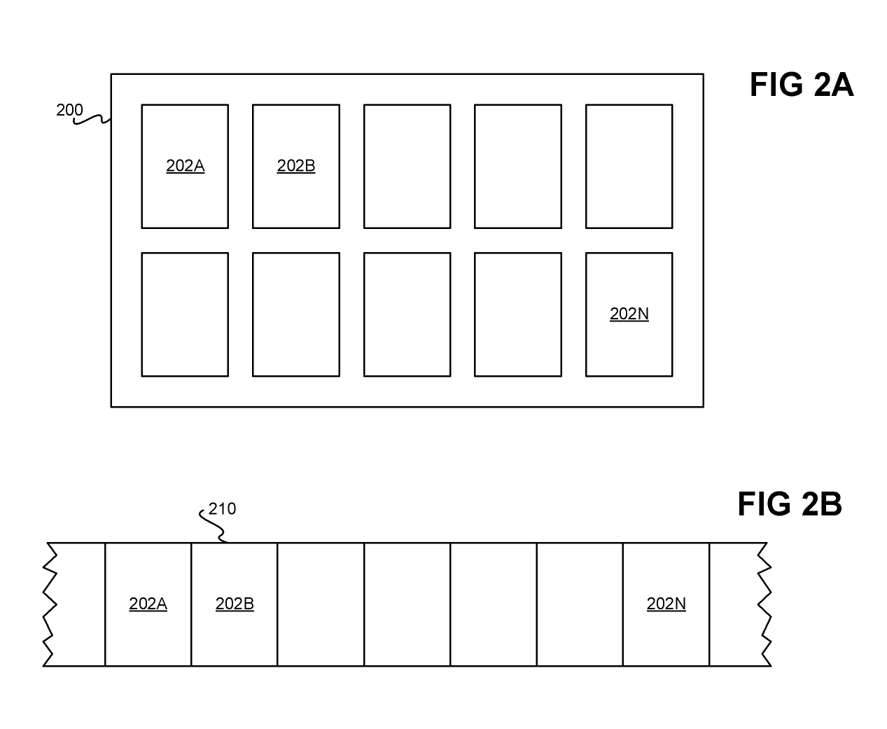

FIGS. 2A and 2B show two different ways in which diagnostic test strips 202A, 202B, . . . , 202N, collectively referred to as the diagnostic test strips 202, can be processed (i.e., manufactured) on a relatively large scale basis, according to varying embodiments of the invention. In FIG. 2A, the test strips 202 are arranged on a plate 200 or contained within a sheet of the substrate material. The test strips 202 on the plate 200 can then have reagents dispensed thereon in a drop-on-demand manner, as is described in more detail later in the detailed description. By comparison, in FIG. 2B, the test strips 202 are separably part of a roll 210. The roll 210 can be a single test strip in width, or have multiple test strips across its width. The roll 210 can be unwound and then rewound so that the test strips 202 thereof can have reagents successively dispensed on them in a drop-on-demand manner, as is described in more detail later in the detailed description.

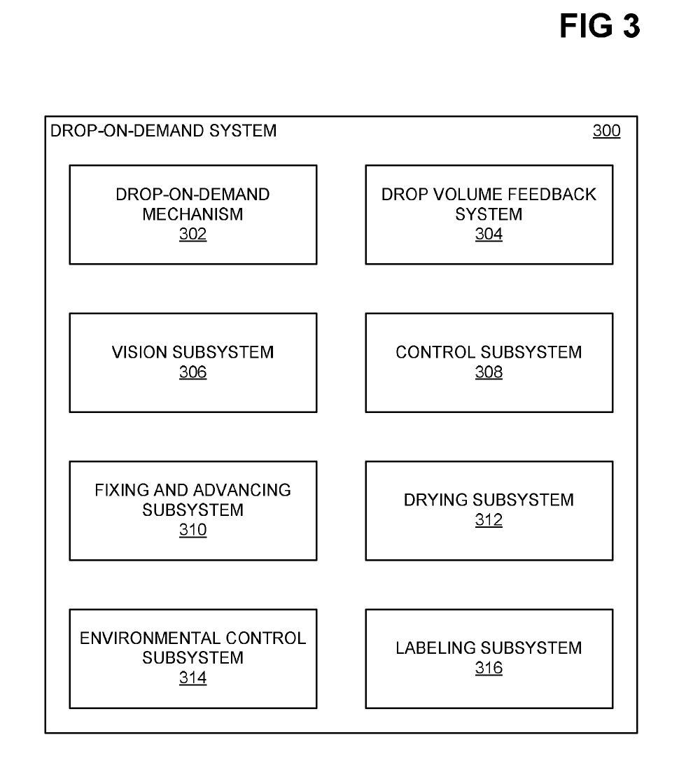

FIG. 3 shows a block diagram of a drop-on-demand system 300 for manufacturing diagnostic test strips, such as the diagnostic test strip 100 of FIG. 1, according to an embodiment of the invention. The drop-on-demand system 300 includes a drop-on-demand mechanism 302, a drop volume feedback subsystem 304, and a vision subsystem 306. The drop-on-demand system 300 also includes a control subsystem 308, a fixing and advancing subsystem 310, a drying subsystem 312, an environmental control subsystem 314, and a labeling subsystem 316. Each of these mechanisms and subsystems 302, 304, 306, 308, 310, 312, 314, and 316 is now described in detail.

The drop-on-demand mechanism 302 is a fluid-ejection mechanism that is capable of ejecting individual droplets of reagent onto diagnostic test strips. That is, the mechanism 302 is capable of dispensing drops of reagent as desired, or “on demand,” onto diagnostic test strips. The drop-on-demand mechanism 302 may be a thermal fluid-ejection mechanism, comparable to a thermal inkjet mechanism or printhead, or a piezoelectric fluid-ejection mechanism, comparable to a piezoelectric inkjet mechanism or printhead, or some other type of drop-on-demand mechanism or printhead. The drop-on-demand mechanism 302 is able to eject droplets of reagent onto diagnostic test strips at levels of precision down to picoliters in size, such that the reagent volume that can be dispensed onto diagnostic test strips is more precise than when micropipettes or screen printing is used.

The drop-on-demand mechanism 302 may contain all the available printing fluid, or may contain only some of the available printing fluid. If the drop-on-demand mechanism just contains a portion of the available fluid, it may be fluidically connected to other devices that contain additional printing fluid. These devices may be mounted near the drop-on-demand mechanism 302, or in some other part of the drop-on-demand system 300. Additionally, the drop-on-demand mechanism 302 may contain pressure-regulating components, or pressure may be regulated by components external to drop-on-demand mechanism 302. The pressure regulation may include the maintenance of a negative pressure at the fluid-ejection nozzles 402 by means of an air path above the fluid level.

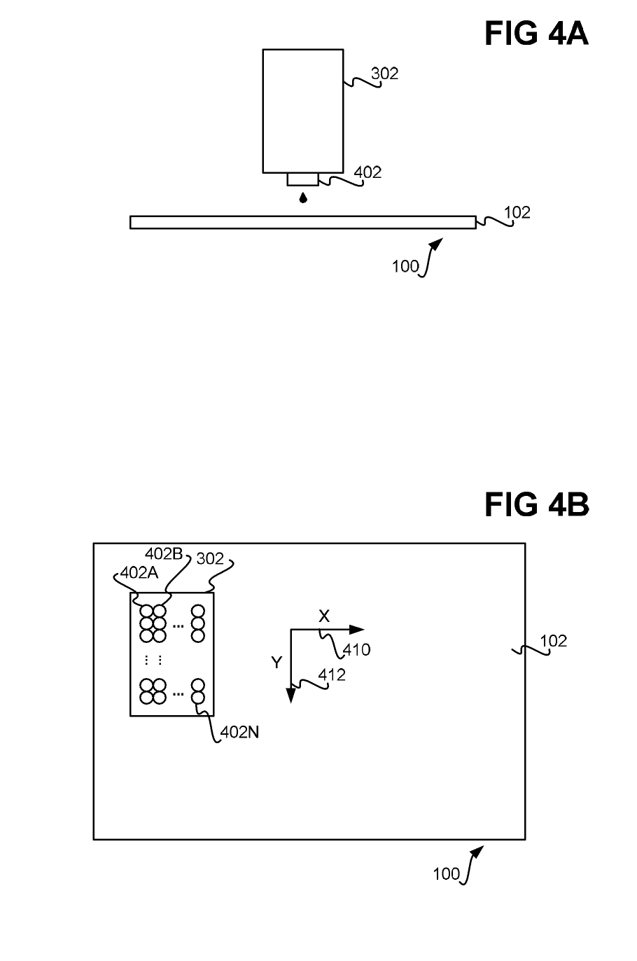

FIGS. 4A and 4B show how the drop-on-demand mechanism 302 can dispense reagent onto the diagnostic test strip 100, according to an embodiment of the invention. In the front view of FIG. 4A, the drop-on-demand mechanism 302 includes a number of fluid-ejection nozzles 402 from which reagent is dispensed onto the substrate 102 of the diagnostic test strip 100. It is noted that no part of the drop-on-demand mechanism 302, including the fluid-ejection nozzles 402, comes into contact with the substrate 102 during reagent dispensing. This is advantageous as compared to when contact printing methods, such screen printing, are used, in which the printing element comes into contact with the diagnostic test strip, since the potential for contamination is greatly reduced.

Such non-contact dispensing also enables the same or different reagents to be placed in very close proximity to one another. Layered reagent structures can be constructed, where droplets of the same or different reagents are placed on top of one another. For instance, layers of reagents can be dispensed in multiple passes of the drop-on-demand mechanism 302 over the diagnostic strip 100, where a thin layer of reagent is dispensed during each pass.

In the top view of FIG. 4B, the drop-on-demand mechanism 302 is depicted for illustrative convenience as being transparent so that the individual fluid-ejection nozzles 402A, 402B, . . . , 402N thereof can be seen. The fluid-ejection nozzles 402 may be organized in aligned or staggered columns and rows. The drop-on-demand mechanism 302 in one embodiment can be moved along both an x-axis 410 and a y-axis 412 in relation to the substrate 102 of the diagnostic test strip 100. As such, more than one of the nozzles 402 can be used to dispense the same or different reagent at the same location on the substrate 102 of the diagnostic test strip 100. In another embodiment, multiple drop-on-demand mechanisms 302, containing the same or different reagents, can be arrayed within a print bar in a moveable or fixed position over the substrate 102 to achieve a higher throughput. For instance, in one embodiment, there may be multiple such print bars, each containing one or more drop-on-demand mechanisms 302, such that the same or different reagents may be disposed onto the substrate 102 at the same time to achieve higher throughput.

For example, in relation to FIG. 1, the reagent 104A has been dispensed at a particular location on the substrate 102 of the diagnostic test strip 100. More than one of the fluid-ejection nozzles 402 of FIGS. 4A and 4B may have dispensed the reagent 104A at this location on the substrate 102. Having more than one fluid-ejection nozzle dispense the same or different reagent at the same location of the substrate 102 of the diagnostic test strip 100 can be advantageous. This is because any potential deficiency in any one of the nozzles 402 is compensated for by having more than one of the nozzles 402 dispense the reagent.

For instance, if a given nozzle is dispensing 10% less than the specified amount of reagent, having this one nozzle dispense all the reagent intended for a given location on the substrate 102 of the diagnostic test strip 100 results in the amount of reagent at this location being 10% less than the intended amount. However, if this nozzle, along with nine other nozzles that each dispense the specified amount of reagent, having all of these ten nozzles dispensing one-tenth of the reagent intended for a given location on the substrate 102 results in the amount of reagent at this location being just 1 % less than the intended amount. In this way, dispensing errors are decreased.

In addition to the approach of reagent dispense depicted in FIG. 4B, other approaches by which the drop-on-demand mechanism 302 dispenses reagents can also be employed. That is, the approach depicted in FIG. 4B involves maintaining the diagnostic test strip 100 in a stationary position while the drop-on-demand mechanism 302 is moved over the diagnostic test strip 100 horizontally and/or vertically. In another approach, however, diagnostic test strips may be moved rapidly past a stationary drop-on-demand mechanism 302 or an array of drop-on-demand mechanisms 302, similar to the way in which industrial inkjet equipment operates (i.e., as a continuous flow, web-based process), as can be appreciated by those of ordinary skill within the art.

Using the drop-on-demand mechanism 302 to dispense reagents on diagnostic test strips is advantageous in a number of different ways, in addition to those that have already been described. One or more reagents can be placed on the diagnostic test strips in complex patterns, due to the presence of multiple fluid-ejection nozzles 402, due to each of these nozzles 402 being able to dispense small amounts of reagent, and due to different sets of the nozzles 402 being able to dispense different reagents. Improvement in placement accuracy of the reagents on diagnostic test strips also results from using the drop-on-demand mechanism 302 due to the inherent droplet dispensing resolution (300-2400 dpi) and the accuracy and precision of motion in the x-axis and y-axis. Due to the improved accuracy and precision in placement and dispensed volume by the drop-on-demand mechanism 302, less overprinting of reagent onto inactive regions of the test strip has to occur, and less reagent can be used on each test strip. Additionally, because of the disposable nature of the drop-on-demand mechanism 302 in some embodiments, no flushing or priming of tubing or building up excess on a screen is required, so there is less reagent wastage.

Utilizing the drop-on-demand mechanism 302 to dispense reagents on diagnostic test strips has been found to be able to be employed even where the reagents have biological molecular bioactivity. For example, although thermal fluid-ejection in particular uses thermal energy (i.e., heat) to eject reagent droplets, little if any degradation of reagent bioactivity occurs due to the high heat-transfer rates and low volume of fluid in contact with the resistor and involved in the bubble nucleation event. In general, therefore, bioactivity retention has been demonstrated when using the drop-on-demand mechanism 302.

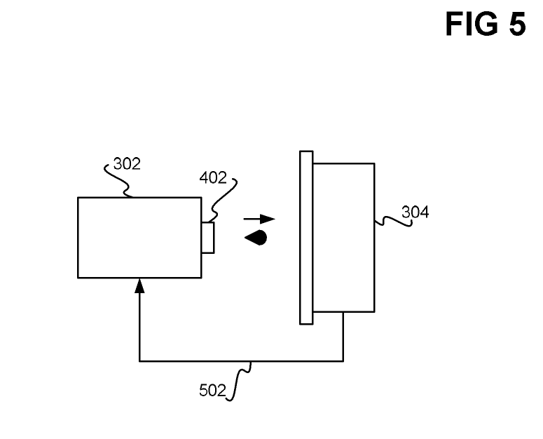

Referring back to FIG. 3, the drop volume feedback subsystem 304 of the drop-on-demand system 300 controls the volumes of reagents dispensed by the drop-on-demand mechanism 302, so that the mechanism 302 dispenses the reagents onto diagnostic test strips in the desired volumes. FIG. 5 shows how the drop volume feedback subsystem 304 can control the volume of reagent dispensed by the drop-on-demand mechanism 302, according to an embodiment of the invention. In general, the subsystem 304 measures the volume of droplets ejected by each of the fluid-ejection nozzles 402 of the drop-on-demand mechanism 302 and compares the volume against the intended droplet volume. In response, as indicated by the arrow 502, the subsystem 304 provides feedback to the fluid-ejection mechanism 302, so that more or less droplets from each fluid-ejection nozzle 402, for instance, can be ejected when dispensing reagent so that the desired volume is ejected. The subsystem 304 is thus a closed-loop feedback system that periodically verifies the volumetric output of reagents by the nozzles 402 of the mechanism 302. It is noted that for thermal inkjet mechanisms, modifying the firing energy does not significantly change drop volume, whereas for piezoelectric inkjet mechanisms, modifying the driving pulse or waveform can change drop volume.

Different approaches can be utilized by the drop volume feedback subsystem 304 to verify volumetric output of reagents by the drop-on-demand mechanism 302. For example, an optical approach can be used, such that the subsystem 304 is an optical subsystem. A number of reagent droplets may be ejected onto a target, which may be a test diagnostic strip itself. The target is then optically scanned to measure the size of the reagent spot resulting from the droplets ejected. If the target is smaller than intended, the drop-on-demand mechanism 302 may increase the number of droplets ejected, for instance, to increase volume. If the target is greater than intended, the mechanism 302 may decrease the decrease the number of droplets ejected, for instance, to decrease volume.

Another approach that can be utilized by the drop volume feedback subsystem 304 to verify volumetric output is a conductivity-based approach, such that the subsystem 304 is a conductivity subsystem. A number of reagent droplets may be ejected onto a target. The conductivity of the size of the reagent spot resulting from the droplets ejected is measured. Based on how much the spot-conductivity deviates from an expected conductivity, the drop-on-demand mechanism 302 can modify the number of droplets ejected, for instance, to dispense reagent droplets, so that the desired volumetric output is achieved.

A third approach that can be utilized by the drop volume feedback subsystem 304 to verify volumetric output is a gravimetric-based approach, such that the subsystem 304 is a gravimetric subsystem. A number of reagent droplets may be ejected onto a sensitive scale, such as a microbalance. The resulting average weight of a reagent droplet is then determined. Based on how much the average weight deviates from an expected average weight, the drop-on-demand mechanism 302 can modify the number of droplets ejected, for instance, to dispense reagent droplets, so that the desired volumetric output is achieved. A fourth approach that can be utilized by the drop volume feedback subsystem 304 to verify volumetric output is a scattering approach, such that the subsystem 304 is a scattering subsystem. A number of reagent droplets may be ejected onto a target, as before. An x-ray, or another type of ray, such as a ray of visible light, is caused to impinge the reagent spot resulting from the droplets ejected. The resulting scattering of the rays is then measured. Based on how much the ray scattering deviates from an expected ray scattering, the drop-on-demand mechanism 302 can modify the number of droplets ejected, for instance, to dispense reagent droplets, so that the desired volumetric output is achieved.

Finally, a fifth approach that can be utilized by the drop volume feedback subsystem 304 to verify volumetric output is an optical absorption, fluorescence, luminescence, or phosphorescence approach. Such an optical approach is described in the patent application entitled Sensing of Fluid Ejected By Drop-On-Demand Nozzles. [attorney docket no. 200603580-1], which is being filed on the same day as the present patent application is. In this approach, the drop volume feedback subsystem 304 could include a well plate reader. A well plate could concurrently receive fluid droplets ejected from a single nozzle or from multiple nozzles or a selected grouping of nozzles. The well plate reader may concurrently detect the one or more characteristics of fluid ejected from the nozzles. The well plate reader is configured to emit and direct light or electromagnetic radiation towards the ejected fluid contained within well plates to sense an optical property of the ejected fluid such as absorbance, fluorescence, phosphorescence, luminescence among others. This sensed information regarding the fluid property is evaluated to determine a volume of the ejected fluid.

As can be appreciated by those of ordinary skill within the art, other approaches can be employed by the drop volume feedback subsystem 304 to verify volumetric output of the reagents dispensed by the drop-on-demand mechanism 302. Such approaches may be performed periodically to ensure that the mechanism 302 is properly ejecting reagents. Besides modifying the number of droplets ejected from each fluid ejection nozzle, the mechanism 302 can specify more or less fluid ejection nozzles to be used. Individual nozzles in mechanism 302 can be turned off and replaced with properly functioning nozzles using software or firmware coupled with mechanism 302 motion control. Volume variations can arise from a number of different causes. A given drop-on-demand mechanism 302 may slowly degrade over its rated lifetime. Environmental conditions, such as temperature and humidity, can affect volumetric output. Changes in the composition and temperature of the reagents themselves can also affect volumetric output.

In one embodiment, the drop volume feedback subsystem 304 can be used to verify volumetric output of the reagents dispensed by the drop-on-demand mechanism 302 before any diagnostic test strips have had reagents dispensed thereon by the mechanism 302. In another embodiment, however, the subsystem 304 is considered an “in use” subsystem that periodically verifies volumetric output after the drop-on-demand mechanism 302 has already started dispensing reagents onto diagnostic test strips. In this embodiment, other approaches are employed to ensure that the mechanism 302 ejects the proper volume of reagents at the beginning of reagent ejection onto test strips. As just one example, the drop-on-demand mechanism 302 may itself be designed so that at the beginning of its life, it is known what volume ejection of reagents therefrom will result as a function of known usage. A known performance over time can also be used to compensate for changes in dispensed volume.

Referring back to FIG. 3, the vision subsystem 306 of the drop-on-demand system 300 aligns the drop-on-demand mechanism 302 in relation to the diagnostic test strips so that the drop-on-demand mechanism 302 dispenses the reagents on the diagnostic test strips at the desired locations, with better accuracy and precision than can be achieved using other methods. For instance, reagents can be placed within ten microns of the desired location. In this way, the vision subsystem 306 compensates for variations in directionality and alignment of the drop-on-demand subsystem 302 in relation to the diagnostic test strips. In this way as well, the vision subsystem 306 compensates for variations within the dimensions of the reagent dispense target region of the diagnostic test strips themselves.

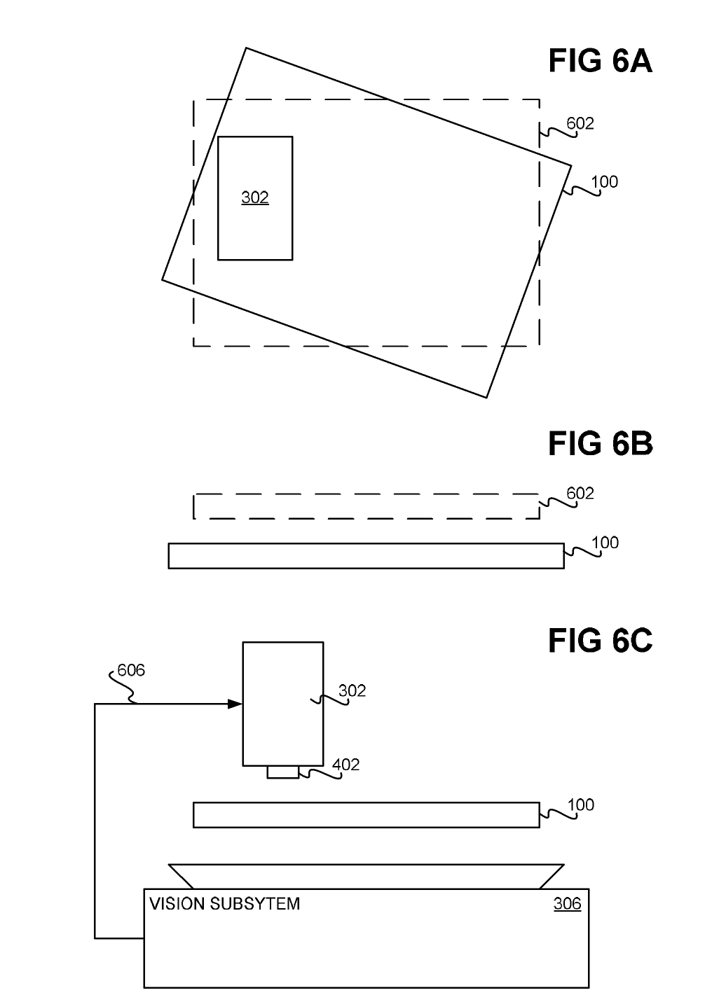

FIGS. 6A, 6B, and 6C show how the vision subsystem 306 can compensate for variations within and between the drop-on-demand mechanism 302 and the diagnostic test strip 100, according to varying embodiments of the invention. In the top view of FIG. 6A, the diagnostic test strip 100 should be positioned relative to the drop-on-demand mechanism 302 as indicated by the dotted-line box 602. However, in actuality, the diagnostic test strip 100 is slightly skewed—specifically rotated clockwise—in relation to its proper position. In the front view of FIG. 6B, the diagnostic test strip 100 should have left and right ends indicated by the dotted-line box 604. However, in actuality, the diagnostic test strip 100 is slightly stretched, such that it has left and right ends that are further out than their proper positions.

Therefore, in the front view of FIG. 6C, the vision subsystem 306 is capable of detecting such variations within and between the drop-on-demand mechanism 302 and the diagnostic test strip 100. In particular, the vision subsystem 306 is a machine vision subsystem, that optically detects the location of the diagnostic test strip in relation to the drop-on-demand mechanism 302 and thus in relation to the test strip's proper position. The vision subsystem 306 provides this information to the drop-on-demand mechanism 302, as indicated by the line 606, so that the mechanism 302 or the strip 100 can be moved, or so that different fluid ejection nozzles can be utilized, or so that the print pattern can be modified, and dispense reagent in relation to the actual position of the diagnostic test strip. The vision subsystem 306 may also determine the location of every test strip to be printed, or the location of just one or a few test strips to be printed out of a larger set of test strips. The vision subsystem 306 may monitor test strips or for other features continuously or periodically.

For example, in relation to the skewing of FIG. 6A, vertical and horizontal movements of the drop-on-demand mechanism 302 can compensate for the actual position of the diagnostic test strip 100. Additionally or alternatively, the pattern to be printed by the drop-on-demand mechanism 302 can be modified based on feedback from the vision system to compensate for the actual position of the diagnostic test strip 100. As such, variations in the directionality and the alignment of the drop-on-demand mechanism 302 in relation to the diagnostic test strip 100 are compensated for by the vision subsystem 306. In relation to the stretching of FIG. 6B, horizontal movement of the drop-on-demand mechanism 302 can be scaled upwards or the pattern to be printed can be modified to accommodate the greater length of the diagnostic test strip 100 and the greater spacing between diagnostic test strips on the substrate 200 or roll 210. As such, variations within the diagnostic test strip 100 or within the substrate 200 or roll 210, or within the print mechanism 302 are compensated for by the vision subsystem 306.

Utilization of the vision subsystem 306 to align the drop-on-demand mechanism 302 in relation to diagnostic test strips can be especially important where there are a number of such diagnostic test strips. For example, in relation to FIG. 2B, the roll 210 includes a large number of diagnostic test strips 202. Aligning the mechanism 302 in relation to these test strips 202 ensures that a large number of diagnostic test strips 202 are not manufactured improperly, which would otherwise result in wastage. Furthermore, variations within the diagnostic test strips 202 such as that depicted in FIG. 6B can be common where the test strips 202 are subject to processing before dispensing of reagents thereon, such that compensation for these variations via the subsystem 306 ensures that the prior processing of the strips 202 is not wasted.

The vision subsystem 306 may optically detect the diagnostic test strips in a number of different ways. As has been alluded to earlier, the vision subsystem 306 may detect the edges of the diagnostic test strips. In another embodiment, however, specific features on or near the diagnostic test strips, or between the test strips, or on the perimeter or margin of the plate 200 or the roll 210, may be optically detected, in addition to or in lieu of optical detection of the edges of the test strips. Such features may be alignment marks or patterns, fiducials, crosses, or other features of the test strips, such as electrode edges, for instance.

The vision subsystem 306 may automatically perform its alignment of the drop-on-demand mechanism 302 in relation to a diagnostic test strip as soon as a new diagnostic test strip has been moved to have reagents dispensed thereon by the mechanism 302. Thus, prior to the drop-on-demand mechanism 302 dispensing reagents on a given diagnostic test strip or set of closely adjacent strips, the vision subsystem 306 determines whether any adjustments should be made to the alignment of the mechanism 302 in relation to the diagnostic test strip, to the print pattern, or to the specific droplet ejection nozzles to be used to generate the print pattern. Once such adjustments have been made, by either moving the test strip, the drop-on-demand mechanism 302, or both, or by modifying the print pattern, the drop-on-demand mechanism 302 or the test strips can be moved in an informed manner, so that proper locational dispensing of the reagents on the diagnostic test strip occurs.

Referring back to FIG. 3, the drop-on-demand system 300 can include other subsystems, in addition to the drop-on-demand mechanism 302, the drop volume feedback subsystem 304, and the vision subsystem 306. For instance, the control subsystem 308 may control the drop-on-demand mechanism 302 to control at least the pattern of the reagents dispensed on the diagnostic test strips. The control subsystem 308 may be part of the same subsystem as the drop volume feedback subsystem 304 in one embodiment, or may be a separate subsystem in another embodiment of the invention, and may work independently or in conjunction with the vision subsystem 304.

The control subsystem 308 may, for example, by specifying various firing parameters of the drop-on-demand mechanism 302, define which of the fluid-ejection nozzles 402 of the mechanism 302 are to be used and in which order, define the image or pattern of the reagents to be dispensed onto the diagnostic test strips, and how this image or pattern is to be realized. The control system may also specify electrical firing parameters or driving pulse or waveform to define the droplet volume ejected out of each nozzle being used. In this way, the control subsystem 308 controls at least the morphology of the reagents dispensed on the diagnostic test strips. The shape aspect of the morphology may be controlled, for example, in that the pattern of the reagents to be dispensed on the diagnostic test strips is controlled. The texture and/or thickness aspect of the morphology may be controlled, for example, in that the number of layers or print pattern of the reagents to be dispensed on the diagnostic test strips is controlled.

The fixing and advancing subsystem 310 of the drop-on-demand system 300 includes those components that advance or move the drop-on-demand mechanism 302 and/or the diagnostic test strips themselves, as well as that can hold, or fix, the test strips in place while the mechanism 302 dispenses reagents thereon. For example, the subsystem 310 may include various motors that wind and unwind the roll 210 of diagnostic test strips so that the diagnostic test strips 202 are moved past the drop-on-demand mechanism 302. As another example, the subsystem 310 may include various motors to move the drop-on-demand mechanism 302 over the diagnostic test strips 202 situated on the plate 200 of FIG. 2A.

Furthermore, the fixing and advancing subsystem 310 can precisely hold, or fix, the diagnostic test strips in place as the drop-on-demand mechanism 302 dispenses reagents on the test strips. For example, the subsystem 310 may include a vacuum that creates negative air pressure against the current diagnostic test strip on which the mechanism 302 is dispensing reagents. As such, the diagnostic test strip is precisely held in place while the drop-on-demand mechanism 302 is dispensing reagents on this diagnostic test strip. The subsystem 310 may further include belts having sprockets, friction rollers, and/or sheet feeders to hold the diagnostic test strip precisely in place.

The environmental control subsystem 314 of the drop-on-demand system 300 includes those components that maintain the desired temperature, humidity, and composition of the environment in which the drop-on-demand mechanism 302 is dispensing reagents on the diagnostic test strips. The drop-on-demand system 300 may be a closed system that is not exposed to external environmental conditions. As such, the subsystem 314 can include heaters, coolers, humidifiers, dehumidifiers, and so on, to precisely control the gas flows, temperature and humidity, as well as other environmental factors, of this closed system. Such precise control of environmental factors ensures that the optimal environment for precise dispensing of reagents on diagnostic test strips is achieved.

The drying subsystem 312 of the drop-on-demand system 300 includes those components that dry the reagents dispensed onto the diagnostic test strips by the drop-on-demand mechanism 302. For example, the drying subsystem 312 may include a conductive heater that heats the reagents dispensed on the diagnostic test strips to accelerate drying thereof. As another example, the drying subsystem 312 may include an evaporative blower that blows air or another gas, which may or may not be heated, over the reagents dispensed on the diagnostic test strips to accelerate drying thereof. The drying sub-system 312 may include other components capable of drying the reagents in other ways, including submitting the reagents to low pressure or low humidity. Drying of the reagents can occur when all the deposition is complete (when the total volume has been dispensed), or at interim points in the deposition, including between print passes or layers.

Finally, the labeling subsystem 316 may include those components, such as conventional inkjet-printing components, to print human or machine-readable information regarding the diagnostic test strips on the diagnostic test strips, either before or after the drop-on-demand mechanism 302 has dispensed reagents on the test strips. Such human or machine-readable information may include control numbers, the identity of the manufacturer of the diagnostic test strips, the commercial trade name of the test strips, and so on. Such human or machine-readable information may further include other information regarding the diagnostic test strips, such as the sensitivity of the strip, reject marks to be used in subsequent manufacturing processes, coding, both overt and covert, to discourage counterfeiting or enable validation, and other information. The machine-readable information may also be readable by a diagnostic test strip test meter in one embodiment of the invention.

Those of ordinary skill within the art can appreciate that the drop-on-demand system 300 can include other subsystems and/or mechanisms, in addition to and/or in lieu of those depicted in FIG. 3. As just one example, a servicing station subsystem may be present that periodically services the drop-on-demand mechanism 302. Such a subsystem may wipe the mechanism 302, cause its fluid-ejection nozzles 402 to spit, to clear the nozzles 402, or use a vacuum pressure to prime the nozzles 402. This subsystem may also periodically assess the presence of droplets from some or all of the fluid-ejection nozzles 402. Other types of subsystems may thus further be included as part of the drop-on-demand system 300 for manufacturing diagnostic test strips.

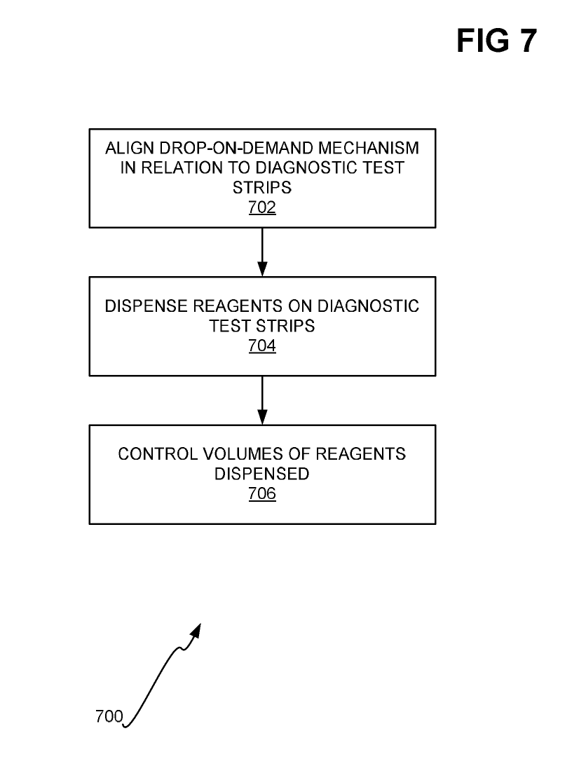

In conclusion, FIG. 7 shows a rudimentary method 700 for manufacturing diagnostic test strips, according to an embodiment of the invention. The steps, parts, or acts 702, 704, and 706 of the method 700 can be performed in any order, and not necessarily in the order depicted in FIG. 7. Furthermore, other steps, parts, or acts, in addition to and/or in lieu of those depicted in FIG. 7, may be part of the method 700, including using part 706 to provide feedback to part 704.

Thus, the vision subsystem 306 aligns the drop-on-demand mechanism 302 in relation to the diagnostic test strips (702). As has been described, this ensures that the drop-on-demand mechanism 302 dispenses reagents on the diagnostic test strips at the desired or proper locations of the test strips. The drop-on-demand mechanism 302 therefore dispenses the reagents on the diagnostic test strips (704). The drop volume feedback subsystem 304 controls the volumes of the reagents dispensed by the mechanism 302 (706), so that the proper volumes of the reagents are dispensed on the diagnostic test strips, as has been described.

We claim:

1. A drop-on-demand system for manufacturing diagnostic test strips, comprising:
a drop-on-demand mechanism to dispense one or more reagents on the diagnostic test strips in one or more desired volumes and patterns at one or more desired locations;
one or more of:
a drop volume feedback subsystem to control volumes of the reagents dispensed by the drop-on-demand mechanism so that the drop-on-demand mechanism dispenses the reagents on the diagnostic test strips in the desired volumes; and,
a vision subsystem to align the drop-on-demand mechanism in relation to the diagnostic test strips so that the drop-on-demand mechanism dispenses the reagents on the diagnostic test strips at the desired locations.

2. The drop-on-demand system of claim 1, wherein the drop volume feedback subsystem comprises a closed-loop feedback subsystem that periodically verifies volumetric output of the reagents by the drop-on-demand mechanism.

3. The drop-on-demand system of claim 1, wherein the drop volume feedback subsystem comprises one or more of an optical subsystem, a conductivity subsystem, a gravimetric subsystem, a scattering subsystem, and an optical absorbance, fluorescence, luminescence, or phosphorescence subsystem.

4. The drop-on-demand system of claim 1, wherein the vision subsystem compensates for variations in alignment and droplet directionality of the drop-on-demand mechanism in relation to the diagnostic test strips.

5. The drop-on-demand system of claim 1, wherein the vision subsystem compensates for variations within or among the diagnostic test strips.

6. The drop-on-demand system of claim 1, wherein the drop-on-demand mechanism comprises a plurality of fluid-ejection nozzles, more than one of the fluid-ejection nozzles capable of dispensing the reagents at multiple of the desired locations on the diagnostic test strips.

7. The drop-on-demand system of claim 1, wherein the drop-on-demand mechanism is a first drop-on-demand mechanism, and the drop-on-demand system further comprises one or more second drop-on-demand mechanisms, such that the first drop-on-demand mechanism and the second drop-on-demand mechanisms constitute a plurality of drop-on-demand mechanisms, the drop-on-demand mechanisms arrayed to efficiently dispense the reagents at each of the desired locations on the diagnostic test strips.

8. The drop-on-demand system of claim 1, wherein the drop-on-demand mechanism does not contact the diagnostic test strips while dispensing the reagents on the diagnostic test strips.

9. The drop-on-demand system of claim 1, wherein the drop-on-demand mechanism is one of a thermal inkjet and a piezoelectric drop-on-demand mechanism.

10. The drop-on-demand system of claim 1, further comprising a control subsystem to control the drop-on-demand mechanism and to at least control a morphology of the reagents dispensed on the diagnostic test strips.

11. The drop-on-demand system of claim 1, further comprising a fixing and advancing subsystem to at least move the diagnostic test strips in relation to the drop-on-demand mechanism and to fix the diagnostic test strips in place while the drop-on-demand mechanism dispenses the reagents on the diagnostic test strips.

12. The drop-on-demand system of claim 1, further comprising an environmental control subsystem to maintain one or more of a desired temperature, a desired humidity, and a desired composition of an environment in which the drop-on-demand mechanism dispenses the reagents on the diagnostic test strips.

13. The drop-on-demand system of claim 1, further comprising a drying subsystem to dry the reagents dispensed on the diagnostic test strips by the drop-on-demand mechanism.

14. The drop-on-demand system of claim 1, further comprising a labeling subsystem to print human or machine-readable information regarding each diagnostic test strip on the diagnostic test strip.

15. A drop-on-demand system for manufacturing diagnostic test strips, comprising:
drop-on-demand means for dispensing one or more reagents on the diagnostic test strips in one or more desired volumes and patterns at one or more desired locations; and,
one or more of:
drop volume feedback means for controlling volumes of the reagents dispensed by the drop-on-demand mechanism so that the drop-on-demand means dispenses the reagents on the diagnostic test strips in the desired volumes; and,
vision means for aligning the drop-on-demand means in relation to the diagnostic test strips so that the drop-on-demand means dispenses the reagents on the diagnostic test strips at the desired locations and with the desired patterns.

16. The drop-on-demand system of claim 15, wherein the drop-on-demand means comprises a plurality of fluid-ejection nozzles, each fluid-ejection nozzle capable of dispensing the reagents at multiple of the desired locations on the diagnostic test strips.

17. The drop-on-demand system of claim 15, wherein the drop-on-demand means is a first drop-on-demand means, and the drop-on-demand system further comprises one or more second drop-on-demand means, such that the first drop-on-demand means and the second drop-on-demand means constitute a plurality of drop-on-demand means, the drop-on-demand means arrayed to efficiently dispense the reagents at each of the desired locations on the diagnostic test strips.

18. The drop-on-demand system of claim 15, wherein the drop-on-demand means does not contact the diagnostic test strips while dispensing the reagents on the diagnostic test strips.

19. The drop-on-demand system of claim 15, wherein the drop-on-demand means is one of a thermal inkjet and a piezoelectric drop-on-demand means.

20. A method comprising:
dispensing one or more reagents on diagnostic test strips, using a drop-on-demand mechanism;
aligning the drop-on demand mechanism in relation to the diagnostic test strips so that the reagents are dispensed on the diagnostic test strips at one or more desired locations and patterns on the diagnostic test strips; and,
controlling volumes of the reagents dispensed so that the reagents are dispensed on the diagnostic test strips in one or more desired volumes, using a drop volume feedback subsystem.
